(19) United States
(12) Patent Application Publication
(10) Pub. No.: US 2025/0186196 A1
(43) Pub. Date: Jun. 12, 2025

(54) PROSTHETIC VALVE, VALVE LEAFLET THEREFOR, AND PREPARATION METHOD THEREFOR

(71) Applicant: VENUS MEDTECH (HANGZHOU) INC., Hangzhou (CN)

(72) Inventors: Dajun Kuang, Hangzhou (CN); Xing Wei, Hangzhou (CN); Zhaogang Liu, Hangzhou (CN); Shasha Xu, Hangzhou (CN)

(21) Appl. No.: 19/059,331

(22) Filed: Feb. 21, 2025

Related U.S. Application Data
(63) Continuation of application No. PCT/CN2023/106861, filed on Jul. 12, 2023.

(30) Foreign Application Priority Data
Aug. 22, 2022 (CN) 202211008225.2

(51) Int. Cl. A61F 2/24; A61L 27/18; A61L 27/50; A61L 27/56

(52) U.S. Cl. CPC A61F 2/2415; A61F 2/2418; A61L 27/18; A61L 27/507; A61L 27/56; A61F 2240/004; A61L 2430/20

(57) ABSTRACT

The present application discloses a prosthetic valve, a valve leaflet therefor, and a preparation method therefor. The valve leaflet includes: a main body, wherein an edge portion of the main body includes a free edge and a fixing edge that are opposite to each other, and the main body is made of a first polymer material; and a first reinforcing edge distributed along the fixing edge, wherein the first reinforcing edge is made of a second polymer material which is configured with a porous structure, and the first polymer material enters the porous structure of the first reinforcing edge and is fixed to the first reinforcing edge.

### CROSS-REFERENCE TO RELATED APPLICATIONS

This application is a continuation application of PCT Application No. PCT/CN2023/106861, filed on Jul. 12, 2023, which claims the priority of Chinese Application No.202211008225.2, filed on Aug. 22, 2022, the entire contents of which are hereby incorporated by reference.

### TECHNICAL FIELD

This application relates to the technical field of medical materials, and in particular to a prosthetic valve, a valve leaflet for prosthetic valve, and a preparation method for valve leaflet.

### BACKGROUND

At present, prosthetic heart valves have received widespread attention and research as products for treating heart valve diseases. Prosthetic heart valves mainly include aortic valves, pulmonary valves, mitral valves, and tricuspid valves, which can replace the function of natural heart valves to control the unidirectional flow of blood, thereby achieving the normal activity of human heart organs.

The common prosthetic heart valves are mainly divided into mechanical valves, biological valves and polymer valves, according to the type of material of the valve leaflet. The mechanical valves have a long service life, but they are mainly implanted through surgical means, causing significant damage to the human body. At the same time, the biocompatibility of the mechanical valve is poor, so long-term use of anticoagulants is necessary, leading to complications. The biological valves are currently widely used and have minimal damage to the human body, but they have problems with short service life and poor tolerance. Therefore, in recent years, the research on polymer valves has received widespread attention, mainly because the characteristics of polymer materials themselves make them have good biocompatibility, long service life, good flexibility, and flexibility to achieve good fluid dynamics performance.

For example, the currently widely used materials for polymer valve leaflets mainly include polyurethane, polyolefin, and polysiloxane, which have been widely used in the preparation of implantable medical devices and have been proven to have excellent high elasticity, good biocompatibility, and flexibility. Compared with biological membranes, polymer valve leaflets further have the advantages of simple processing technology, uniform thickness and performance for mass production. However, with the discovery of research, it has been found that when the polymer valves are sutured to valve frames, the valve leaflets may encounter uneven stress and tear easily, which seriously limits the development of the polymer valves. Therefore, how to improve the tear resistance of the polymer valve leaflets through structural optimization and innovative design is currently one of the most severe issues in achieving the development of the polymer valves.

### SUMMARY

Based on this, a prosthetic valve, a valve leaflet for prosthetic valve, and a preparation method for valve leaflet are provided to solve the technical problem of poor tear resistance of the polymer valve.

This application provides a valve leaflet for prosthetic valve, including:
a main body, an edge portion of the main body including a free edge and a fixing edge that are opposite to each other, the main body being made of a first polymer material; and
a first reinforcing edge distributed along the fixing edge, the first reinforcing edge being made of a second polymer material which is configured with a porous structure, the first polymer material entering the porous structure of the first reinforcing edge and being fixed to the first reinforcing edge.

Several optional embodiments are provided below, which are only further supplements or optimizations and do not serve as additional limitations on the above overall solution. Under the premise of no technical or logical contradictions, each optional embodiment may be combined separately for the above overall solution, or the optional embodiments may be combined with each other.

Optionally, the valve leaflet for prosthetic valve further includes:
a second reinforcing edge distributed along the free edge, the second reinforcing edge being made of a third polymer material which is configured with a porous structure, the first polymer material entering the porous structure of the second reinforcing edge and being fixed to the second reinforcing edge.

Optionally, the first reinforcing edge and the second reinforcing edge are made of the same material.

Optionally, the first reinforcing edge and the second reinforcing edge are integrally formed as one piece.

Optionally, the second reinforcing edge in whole is substantially strip-shaped and extends along the free edge with a constant width.

Optionally, the first reinforcing edge extends outward from a position thereof adjacent to the free edge to form a hanging ear that is sutured with a stent.

Optionally, the first reinforcing edge is configured as a sheet, with a uniform thickness of 0.01-0.3 mm; and, the second reinforcing edge is configured as a sheet, with a uniform thickness of 0.01-0.3 mm.

Optionally, the first reinforcing edge is configured as a sheet, with a uniform thickness of 0.05˜0.2 mm; and, the second reinforcing edge is configured as a sheet, with a uniform thickness of 0.05˜0.2 mm.

Optionally, the first reinforcing edge and the second reinforcing edge have the same thickness.

Optionally, the valve leaflet includes a first region avoiding the first reinforcing edge and/or second reinforcing edge, and a second region overlapping the first reinforcing edge and/or second reinforcing edge, and the first and second regions are substantially the same in thickness.

Optionally, a thickness of the valve leaflet is 0.07˜0.5 mm.

Optionally, the first polymer material is at least one of polyurethane, polyolefin, and polysiloxane.

Optionally, the second polymer material is in form of a fabric, and a raw material of the second polymer material is one of polyester, aramid, nylon, silk, and polyurethane.

Optionally, the third polymer material is in form of a fabric, and a raw material of the third polymer material is one of polyester, aramid, nylon, silk, and polyurethane.

Optionally, the fabric is non-woven fabric or woven fabric.

Optionally, the fabric is a woven fabric with a weft knitted structure and a porosity of 30-80%.

Optionally, the first polymer material is polyurethane or polyolefin; the second polymer material is polyester or nylon; and the third polymer material is polyester or nylon.

Optionally, the first polymer material is polyurethane; and the second and third polymer materials both are polyester.

Optionally, the first polymer material, the second polymer material, and the third polymer material are prepared using the same type of monomer.

Optionally, the first polymer material is polyurethane, with a number average molecular weight of 30000-200000 and a hard segment content of 35-50%; and,
the second polymer material and the third polymer material both are polyurethane, with a number average molecular weight of 80000-250000 and a hard segment content of 45-60%, and the second polymer material and the third polymer material both are configured in form of non-woven fabric and prepared by electrostatic spinning.

Optionally, the first polymer material is polyurethane, with a number average molecular weight of 70000 and a hard segment content of 42%; and,
the second polymer material and the third polymer material both are polyurethane, with a number average molecular weight of 12000 and a hard segment content of 52%, and the second polymer material and the third polymer material both are configured in form of non-woven fabric and prepared by electrostatic spinning.

This application further provides a preparation method for valve leaflet and a valve leaflet obtained by the preparation method.

The preparation method includes:
providing a reinforcing material configured in sheet, the reinforcing material having a porous structure and a reserved region;
compositing a first polymer material in flowing state with the reinforcing material; and
solidifying the first polymer material, and cutting the reinforcing material and/or solidified first polymer material according to a predetermined shape to obtain a valve leaflet, wherein a portion of the valve leaflet with the reinforcing material is a reinforcing edge.

Optionally, the reserved region is a hollow portion of the reinforcing material, and an edge of the hollow portion is partially open or a closed curve.

Optionally, the first polymer material in flowing state is in solution state or molten state, and has a viscosity of 0.4-2.0 Pa·s.

Optionally, the first polymer material in molten state has a melting temperature of 150-300° C.

Optionally, when the edge of the hollow region is closed curve, the reinforcing material including a first reinforcing region and a second reinforcing region enclosing the reserved region, wherein
the first reinforcing region is configured with one side thereof facing the reserved region and corresponding to the fixing edge of the valve leaflet and another side thereof extending away from the reserved region; and
the second reinforcing region is configured with one side thereof facing the reserved region and corresponding to the free edge of the valve leaflet and another side thereof extending away from the reserved region.

Optionally, the first reinforcing region and the second reinforcing region are woven together using the second polymer material and then cut out to form the reserved region; or, the first reinforcing region and the second reinforcing region are woven together and avoided the reserved region during the weaving process.

This application further provides a prosthetic valve that includes:
a stent being configured with a blood flow channel therein;
one or multiple valve leaflet(s) provided by the present application, wherein the first reinforcing edge of the valve leaflet is fixed to the stent, and the free edge of the valve leaflet cooperates with an inner wall of the stent or the free edges of the multiple leaflets cooperate with each other to control the blood flow channel.

The prosthetic valve leaflet provided in this application has good blood compatibility, high anti-tearing strength, and long service life. The effective orifice area and regurgitation ratio of the prosthetic valve manufactured from the valve leaflet are better.

### BRIEF DESCRIPTION OF THE DRAWINGS

FIG. 1 is a schematic view of a molding mold.

FIG. 2 is a schematic view of a fabric after cutting, being configured with a reserved region.

FIG. 3 is a schematic view of a fabric after cutting, being configured with a reserved region.

FIG. 4 is a schematic view of a fabric after cutting, being configured with a reserved region.

FIG. 5 is a schematic view of a valve leaflet configured with a first reinforcing edge.

FIG. 6 is a schematic view of a fabric after cutting, being configured with a reserved region.

FIG. 7 is a schematic view of a valve leaflet being configured with a first reinforcing edge and a second reinforcing edge.

FIG. 8 is a view showing a test result of platelet adsorption.

In the drawings:
1, valve leaflet 1; 11, main body;
2, reinforcing edge; 21, first reinforcing edge; 22, second reinforcing edge;
31, free edge; 32, fixing edge;
5, reinforcing material; 51, first reinforcing region; 52, second reinforcing region;
6, reserved region;
7, mold; 71, settlement region.

### DESCRIPTION OF THE EMBODIMENTS

Technical solutions of embodiments of the present application will be clearly and completely described below in combination with the drawings. Obviously, the described embodiments are only part of, rather than all of the embodiments of the present application. Based on the embodiments of this application, all other embodiments obtained by those ordinary skilled in the art without any creative work shall fall within the protection scope of this application.

In order to better describe and illustrate the embodiments of the present application, reference may be made to one or more figures, but the additional details or examples used to describe the figures should not be construed as limiting the scope of the application, the embodiments currently described, or the preferred embodiments of the present application.

It should be noted that when a component is referred to as “connected” to another component, it may be directly connected to another component or there may be a middle component connected therebetween. When a component is considered to be “set on” another component, it may be directly set on another component or there may be a middle component arranged therebetween.

Unless defined otherwise, all technical and scientific terms used herein have the same meaning as commonly understood by one of ordinary skill in the art to which this application belongs. The terms used in the specification of the present application are only for the purpose of describing specific embodiments and are not intended to limit the present application.

Referring to FIG. 5, a valve leaflet 1 for prosthetic valve includes:
a main body 11, an edge portion of the main body 11 including a free edge 31 and a fixing edge 32 that are opposite to each other, the main body 11 being made of a first polymer material; and
a first reinforcing edge 21 distributed along the fixing edge 32, the first reinforcing edge 21 being made of a second polymer material and configured with a porous structure, the first polymer material entering the porous structure of the first reinforcing edge 21 and being fixed to the first reinforcing edge 21.

The fixing edge 32 of the main body 11 is generally the part through which the prosthetic valve is sutured by sutures. Due to the presence of pinholes, the fixing edge 32 is more prone to tearing. In this application, a first reinforcing edge 21 is provided at the fixing edge 32 of the main body 11 of the valve leaflet 1, which means that the polymer valve leaflet 1 is reinforced in the easily torn and/or stress concentrated areas, thereby improving the anti-tearing strength of the valve leaflet 1 and extending its service life. At the same time, no reinforcement is made at the free edge 31 of the main body 11, so as to ensure the flexibility of the valve leaflet 1 and the sensitivity of opening/closing blood flow channel.

The first reinforcing edge 21 is distributed along the fixing edge 32, which improves the strength of the fixed edge 32. When using suture perforation and connecting the fixing edge 32 to a stent, the first reinforcing edge 21 can improve the strength, fatigue resistance and tear resistance of the connecting portion while ensuring the sensitivity of the free edge 31.

The first polymer material fills into the porous structure of the second polymer material. After solidification, the first polymer material and the reinforcing material are tightly combined in the microstructure, making the fixing relationship between the first polymer material and the reinforcing material 5 tighter and the bonding strength stronger.

The first reinforcing edge 21 in whole is generally strip-shaped and extends along the fixing edge 32 with a constant width; or,
the first reinforcing edge 21 in whole is generally strip-shaped and extends along the fixing edge 32 with a width changed gradually, wherein the width of the first reinforcing edge 21 is max at a midpoint of the fixing edge 32 and gradually narrows towards an intersection of the first fixing edge 32 and the free edge 31.

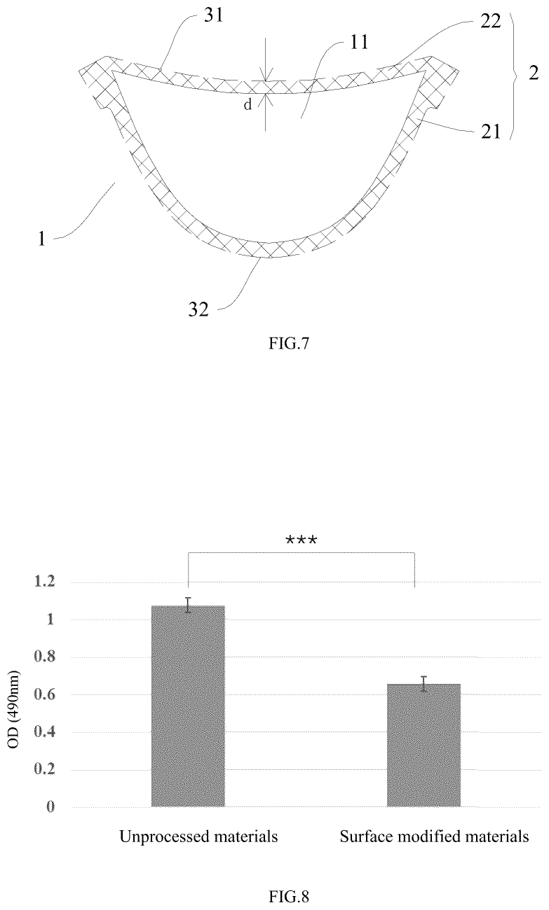

Referring to FIG. 7, the valve leaflet 1 for prosthetic valve further includes:
a second reinforcing edge 22 distributed along the free edge 31, the second reinforcing edge 22 being configured with a porous structure, the first polymer material entering the porous structure of the second reinforcing edge 22 and being fixed to the second reinforcing edge 22.

The second reinforcing edge 22 is made of a third polymer material, but of course, the second reinforcing edge 22 may be made of the same material as the first reinforcing edge 21. It is further preferred that the first reinforcing edge 21 and the second reinforcing edge 22 are integrally formed as one piece.

Similarly, the second reinforcing edge 22 in whole is generally strip-shaped and extends along the free edge 31 with a constant width.

During the operation of the prosthetic valve, the free edge 31 of the valve leaflet 1 moves continuously to close or open blood flow channel of the prosthetic valve. In order to further improve the fatigue resistance of the free edge 31, the second reinforcing edge 22 may be used to reinforce the free edge 31.

At a position adjacent to the free edge 31, the first reinforcing edge 21 extends outward to form a hanging ear that is sutured with the stent. When there is a second reinforcing edge 22, the hanging ear extends approximately from an intersection of the first reinforcing edge 21 and the second reinforcing edge 22. The extension of the first reinforcing edge 21 outward is understood to extend away from a central area of the main body 11, for example, extend upward and/or further to the left and right sides shown in FIG. 7.

Referring to FIG. 7, the width of the second reinforcing edge 22 (indicated by d in the figures) is 0.1-5 mm. Generally, the stress at a center hole of the valve (i.e., position d) is relatively high. By means of reinforcing the free edge 31, the risk of tearing of the valve leaflet 1 is reduced. At the same time, due to the small width of the reinforcement portion, it will not affect the flexibility of the valve leaflet 1.

When suturing the valve leaflet 1 onto the stent, it is even more important to ensure the connection strength between the hanging ear and the stent, so as to ensure the effectiveness of the free edge 31 in controlling blood flow. The hanging ear has two materials, i.e., one for the main body 11 and another for the reinforcing edge 2.

In the present application, without special declaration, the reinforcing edge 2 should be understood as either the first reinforcing edge 21 or the second reinforcing edge 22. Moreover, the material, size, and other physical and chemical properties of the first reinforcing edge 21 and the second reinforcing edge 22 may be configured independently. When the first reinforcing edge 21 and the second reinforcing edge 22 are integral, the reinforcing edge 2 may be understood as the commonality of them.

The reinforcing edge 2 is configured as a sheet and has a uniformly thickness. The thickness of the reinforcing edge 2 is 0.01-0.3 mm, preferably 0.05˜0.2 mm. Excessive thickness of the reinforcing edge 2 is not conducive to obtaining smaller compression dimensions. Especially, increasing hardness at the second reinforcing edge 22 will reduce the sensitivity of the opening and closing of the valve leaflet 1.

The thickness of each portion of the reinforcing edge 2 is generally the same, so as to avoid stress concentration caused by different thicknesses.

The valve leaflet 1 includes a first region that avoids the reinforcing edge 2 and a second region that overlaps with reinforcing edge 2, wherein the thicknesses of the first and second regions are generally the same.

For the case where only the first reinforcing edge 21 is provided, the valve leaflet 1 includes a first region that avoids the first reinforcing edge 21 and a second region that overlaps with the first reinforcing edge 21, wherein the thicknesses of the first and second regions are generally the same.

For the case where the first reinforcing edge 21 and the second reinforcing edge 22 are provided simultaneously, the valve leaflet 1 includes a first region that avoids the first reinforcing edge 21 and second reinforcing edge 22, and a second region that overlaps with the first reinforcing edge 21 and second reinforcing edge 22, wherein the thicknesses of the first and second regions are generally the same.

The thicknesses of the first and second regions are the same, which can avoid loading obstacles caused by excessive thickness of the reinforcing edge 2. After the entire prosthetic valve is compressed, there will be no significant difference in the outer diameter of its various portions.

The thickness of the valve leaflet 1 is 0.01˜0.5 mm. Furthermore, the thickness of the valve leaflet 1 is 0.07-0.5 mm.

Due to the distribution of polymer materials and the infiltration and blending with the reinforcing edge 2, the first and second regions are configured as an integrated structure. As for the main body 11 itself, it may be formed by casting the first polymer material or other methods.

In an ideal state, the thickness of the valve leaflet 1 is the same everywhere, that is, the valve leaflet 1 is generally configured with uniform thickness. During the preparation process, slight differences caused by process conditions may be ignored, and in this case the valve leaflet 1 is still considered to be configured with uniform thickness.

The first polymer material is at least one of polyurethane, polyolefin, and polysiloxane.

The first polymer material is usually a material that can be cast or hot pressed. As a material for the main body 11, the first polymer material needs to meet the basic conditions of being a valve leaflet 1, such as sufficient strength and creep resistance.

The first polymer material is an elastic material, which means that after solidification, it has appropriate elasticity and stronger fatigue resistance, so as to meet the performance requirements of the valve leaflet 1.

The second polymer material and the third polymer material both are configured in the form of fabrics, and their raw materials are independently one of polyester, aramid, nylon, silk, or polyurethane.

The fabric is non-woven fabric or woven fabric. Non-woven fabric may be prepared using electrostatic spinning technology, while woven fabric may be woven using methods such as warp and weft weaving, warp knitting, or weft knitting.

Preferably, the fabric is a weft knitted structure with a porosity of 30-80%. The necessary porosity may ensure the connection strength after composite. A diameter D of the fabric thread is mainly related to the thickness H of the valve leaflet 1. In the thickness direction, the fabric thread overlaps into N layers, wherein N*D is less than or equal to H, for example, at least 50% of H.

The first polymer material and the material of the reinforcing edge 2 need to have good biocompatibility and material compatibility with each other. In the tear prone portion of the valve leaflet 1, the material of the reinforcing edge 2 plays a reinforcing role to improve the creep resistance and edge tear resistance of the valve leaflet 1.

The first polymer material is preferably polyurethane or polyolefin; and, the second polymer material and the third polymer material are each preferably polyester or nylon.

For example, the first polymer material is polyurethane, and the second and third polymer materials both are polyester.

The first polymer material, the second polymer material, and the third polymer material are prepared using the same type of monomer.

The materials prepared using the same type of monomer may have different molecular weights, viscosities, or segment structures. For example, the first polymer material is polyurethane, with a number average molecular weight of 30000-200000 and a hard segment content of 35-50%;
the second and third polymer materials both are polyurethane, with a number average molecular weight of 80000-250000 and a hard segment content of 45-60%. The second and third polymer materials both are configured in form of non-woven fabric and made by electrostatic spinning.

Polyurethane is prepared by the reaction of isocyanates and polyhydroxy compounds. The hard segment mainly refers to the isocyanate residues after the reaction, and in some cases, the hard segment also includes small molecule chain extenders.

The first polymer material is polyurethane, with a number average molecular weight of 70000 and a hard segment content of 42%;
the second and third polymer materials both are polyurethane, with a number average molecular weight of 120000 and a hard segment content of 52%. The second and third polymer materials both are configured in form of non-woven fabric made by electrostatic spinning.

The present application further provides a preparation method for valve leaflet 1, including:
providing a reinforcing material 5 configured as a sheet, the reinforcing material 5 having a porous structure and a reserved region 6, referring to FIG. 2 to FIG. 4;
compositing a first polymer material in flowing state with the reinforcing material 5; and
solidifying the first polymer material, cutting the reinforcing material 5 and/or solidified first polymer material according to a predetermined shape to obtain the valve leaflet 1, wherein a portion of the valve leaflet 1 configured with the reinforcing material is the reinforcing edge.

The reinforcing material is understood as either the second polymer material or the third polymer material. Generally, the second polymer material and the third polymer material are the same, that is, the reinforcing material. In special cases, if the second polymer material and the third polymer material are different materials, they are collectively referred to as the reinforcing material. A portion where the first polymer material is composited with the reinforcing material 5 corresponds to the reinforcing edge 2.

The step of compositing the first polymer material in flowing state with the reinforcing material 5, including at least one of the following operations:
a) making the first polymer material in flowing state have an expected thickness, and immersing the reinforcing material 5 into the first polymer material;
b) dripping and/or coating the first polymer material onto the reinforcing material 5.

In the process of compositing the first polymer material in flowing state with the reinforcing material 5, the first polymer material in flowing state is distributed to the reserved region 6 and enters the porous structure of the reinforcing material 5 around the reserved region 6. The first polymer material fills into the porous structure of the second polymer material, and after solidification, the first polymer material and the reinforcing material are tightly combined in the microstructure, making the fixing relationship between the first polymer material and the reinforcing material 5 tighter and the bonding strength stronger.

The reserved region 6 is a hollow portion of the reinforcing material 5. When only the first reinforcing edge 21 is provided, an edge of the hollow portion may be partially open (referring to FIG. 4), for example, on one side of the free edge 31.

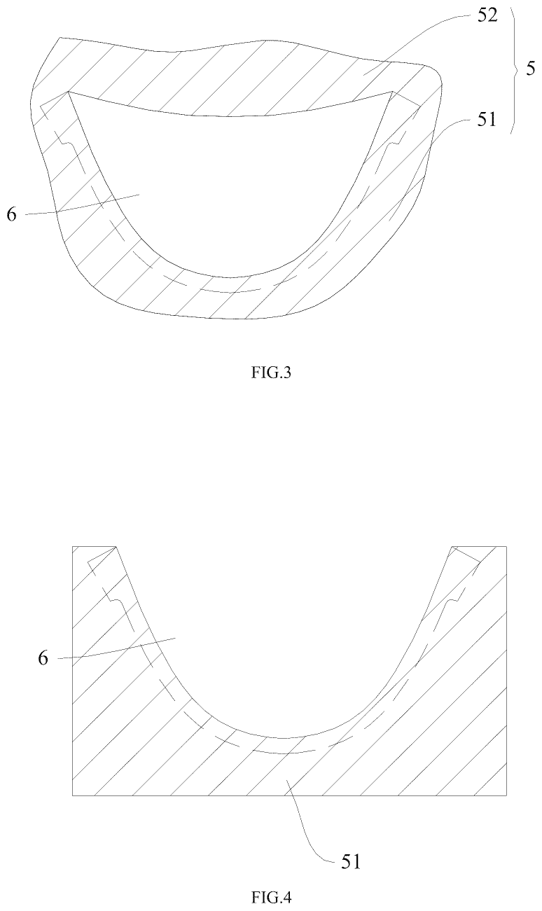

Preferably, the edge of the hollow portion is a closed curve (referring to FIG. 2 and FIG. 3). A portion of the edge of the hollow portion corresponds to the fixing edge 32 of the main body 11, and this portion forms the first reinforcing edge 21 after processing; another portion of the edge of the hollow portion corresponds to the free edge 31 of the main body 11, and this portion forms the second reinforcing edge 22 after processing. The first reinforcing edge 21 and the second reinforcing edge 22, both before and after composite, are integral.

In FIG. 2 to FIG. 4, the blank area is the reserved region 6, the shaded area is the reinforcing material 5, and the dashed line corresponds to the edge of the valve leaflet 1. After the solidification of first polymer material, the valve leaflet 1 can be obtained by cutting along the dashed line. As shown in FIG. 3, the shape of an outer edge of the reinforcing material 5 is not limited, which may be common square, diamond, circular, or irregular, as long as it is similar to the shape of the settlement region of the mold.

Referring to FIG. 4, the shape of the reserved region 6 is not limited to corresponding to the shape of the valve leaflet 1. It is sufficient that the reserved region 6 can form the valve leaflet 1 in predetermined shape through cutting.

When the first polymer material is distributed to the reserved region 6, the first polymer material in flowing state can be coated or injected into the reserved region 6, filling the reserved region 6 and completely immersing the fabric pores of the reinforcing material 5.

For example, the reinforcing material 5 is placed horizontally, and the first polymer material is distributed into the reserved region 6 after an outer circumference of the reserved region 6 is fixed.

When fixed, the reinforcing material 5 may be clamped up and down. Due to the flowing state of the first polymer material, it can penetrate into the clamped portions. By means of fixing the reinforcing material 5 by clamping, it can ensure positioning effect and obtain accurate expected thickness.

Preferably, a portion corresponding to the entire reinforcing edge 2 is clamped. To promote the penetration of the first polymer material, a vacuum may be applied on a periphery of the clamped portion (i.e., a side away from the reserved region 6).

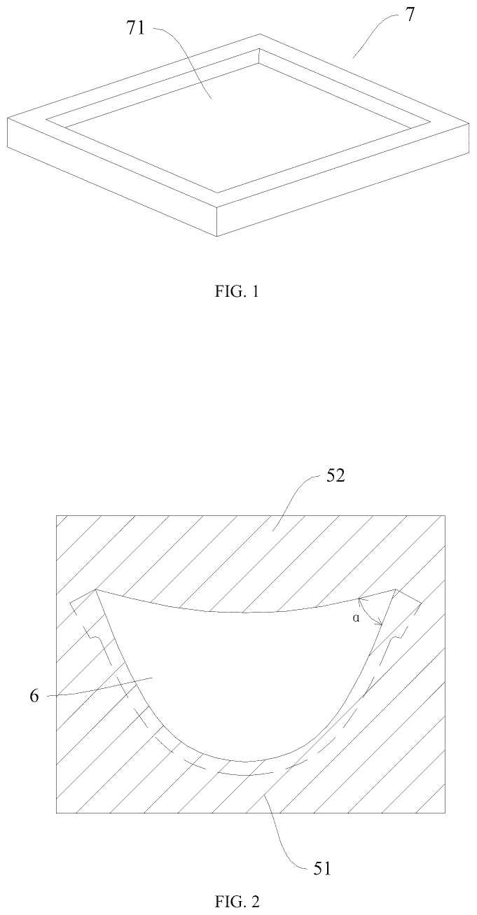

The above process may be achieved using a mold 7. For example, the reinforcing material 5 is placed in the mold 7 and the first polymer material in flowing state is poured into the mold 7, wherein a structure of the mold 7 is shown in FIG. 1.

The mold 7 may be made of materials such as tetrafluoroethylene and stainless steel, with a settling zone 71 being provided on a surface thereof for the laying of the reinforcing material 5 and the leveling of the first polymer material. A depth of settling zone 71 is not strictly limited, and the thickness of the valve leaflet 1 may be calculated based on the amount of material used.

As shown in FIG. 2, when only the first reinforcing edge 21 is provided, the free edge 31 of the valve leaflet 1 is entirely formed by the solidified first polymer material after cutting, that is, the free edge 31 of the valve leaflet 1 does not have the reinforcing material 5 (referring to FIG. 5).

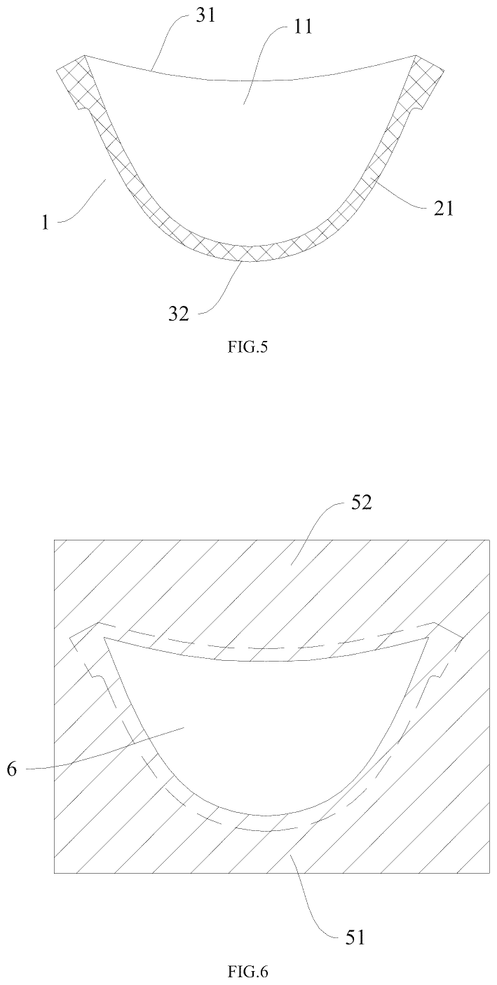

As shown in FIG. 6, when both the first reinforcing edge 21 and the second reinforcing edge 22 are provided, after cutting along the dashed line in FIG. 6, the free edge 31 of the valve leaflet 1 has the first reinforcing edge 21, and the fixing edge of the valve leaflet 1 has the second reinforcing edge 22, wherein a structure of the valve leaflet 1 is shown in FIG. 7.

As shown in FIG. 7, the width at position d is 0.1-5 mm. Generally, the stress at a center hole of the valve (i.e., position d) is relatively high. Thus, reinforcing the free edge 31, the risk of tearing of the valve leaflet 1 is reduced. Due to the small width of the reinforcement portion, it will not affect the flexibility of the valve leaflet 1.

The first polymer material in flowing state includes solution state or molten state, and has a viscosity 0.4-2.0 Pa·s, so as to ensure sufficient leveling of the first polymer material in the reserved region 6 and sufficient penetration of the first polymer material into the fabric pores of the reinforcing material 5.

The mass fraction of the first polymer material in solution state is 1-20%. The solvent may be at least one of N′N-dimethylacetamide, N′N-dimethylformamide, dimethyl sulfoxide, cyclohexane, xylene, or a mixed solvent of at least one of the above solvent with acetone, toluene, tetrahydrofuran, and etc.

A preferred solvent for polyurethane is N′N-dimethylacetamide; a preferred solvent for polyolefins is cyclohexane; and a preferred solvent for polysiloxane is xylene.

The first polymer material in molten state has a melting temperature of 150-300° C.

When the first polymer material in solution state solidifies, it adopts the manner of solvent evaporation, that is, forming a solid structure through simple solvent evaporation.

When the first polymer material in molten state solidifies, cooling is used to obtain a fixed shape for the first polymer material.

After the solidification of the first polymer material, the valve leaflet 1 in predetermined shape may be obtained by laser cutting. Further anticoagulant modification may be performed on a surface of the valve leaflet 1 to improve its blood compatibility.

In order to obtain a valve leaflet 1 with uniform thickness, the following two steps are alternately performed 1-10 times, and then cut according to the predetermined shape:
a) compositing the first polymer material in flowing state with the reinforcing material 5;
b) solidifying the first polymer material.

Each time includes implementing step a) and step b) once each.

This can avoid adverse consequences such as uneven thickness or wrinkling caused by one-time solidifying.

When the edge of the hollow portion is a closed curve, the reinforcing material 5 includes a first reinforcing region 51 and a second reinforcing region 52 enclosing the reserved region 6, wherein
the first reinforcing region 51 is configured with one side thereof facing towards the reserved region 6 and corresponding to the fixing edge 32 of the valve leaflet 1, and another side thereof extending away from the reserved region 6; and
the second reinforcing region 52 is configured with one side thereof facing towards the reserved region 6 and corresponding to the free edge 31 of the valve leaflet 1, and another side thereof extending away from the reserved region 6.

The first reinforcing region 51 and the second reinforcing region 52 cooperatively enclose the reserved region 6. When cutting according to the predetermined shape, the second reinforcing region 52 is cut off, so that only the first polymer material is present at the free edge 31. The first reinforcing region 51 may either correspond exactly to the first reinforcing edge 21 (i.e., its outer periphery does not need to be cut), or be further expanded relative to the first reinforcing edge 21, wherein the expanded area, which is more conducive to clamping and fixing, as well as the diffusion of the first polymer material, reducing the requirements for processing accuracy and technology, needs to be cut.

As shown in FIG. 2, the hollow portion, corresponding to a junction of the free edge and the fixing edge, is configured with an acute angle. During the casting process of the first polymer solution, edges and corners are prevented from curling, ensuring that the two materials are adhered together, thereby greatly improving the yield rate. According to different valve leaflet 1s for the valves in different models, specifications, and designs, fabrics with different sizes and shapes of hollow portions need to be prepared.

As shown in FIG. 2, although the second reinforcing region 52 is ultimately cut off, its presence during the molding process can ensure the overall flatness of the first reinforcing region 51, especially in the areas on two sides of the free edge 31, avoiding local wrinkles and adverse internal stresses. Of course, a portion of the second reinforcing region 52 may be retained to form the second reinforcing edge 22 in the composite process.

As shown in FIG. 2 and FIG. 3, the first reinforcing region 51 and the second reinforcing region 52 may be woven together using the second polymer material and then cut to form the reserved region 6; or, the first reinforcing region 51 and the second reinforcing region 52 may be woven together and avoided the reserved region 6 during the weaving process.

When the first reinforcing region 51 and the second reinforcing region 52 uses integrated woven material 5, laser cutting can be used to cut, wherein a cutting range may be adjusted according to the shape and size of the vale leaflet 1, thereby obtaining the expected shape of the reserved region 6.

In one embodiment, the preparation method of the valve leaflet 1 includes the following steps:
1) dissolving a certain amount of polyurethane (first polymer material) in N,N-dimethylacetamide solution, heating to 80° C. and stirring for 12 hours, fully dissolving the polyurethane to obtain a polyurethane solution with a mass concentration of 12%;
2) cutting a PET fabric (second polymer material) into corresponding sizes through a laser cutting machine (as shown in FIG. 2), placing the cut fabric in a mold and pouring the polyurethane solution into the mold; placing the mold in a blast drying oven to dry it at a temperature of 60 degrees for 12 hours;
repeating the above steps five times to obtain a polymer composite film with a thickness of 0.15 mm;
3) locating a cutting position of a cutting machine accurately to obtain locally reinforced valve leaflet 1, wherein a structure of the valve leaflet 1 is shown in FIG. 5, and the locally reinforced first reinforcing edge 21 is located at a suture portion of the valve leaflet 1.

The performances of locally reinforced valve leaflet 1 prepared using the method described in the above embodiment and non-reinforced valve leaflet 1 (prepared under the same conditions except for absence of PET fabric) is shown in Table 1.

TABLE 1

| | | Tensile | Elongation | Young's | Suture |
|---|---|---|---|---|---|
| | Thickness | Strength | at | Modulus | Force |
| Name | (mm) | (MPa) | Break(%) | (MPa) | (N) |
| reinforced | 0.15 | 43 | 46.5 | 101 | 8.86 |
| valve leaflet 1 | | | | | |
| non-reinforced | 0.15 | 26 | 681 | 10.85 | 5.17 |
| valve leaflet 1 | | | | | |

It can be seen from Table 1, compared to the non-reinforced valve leaflet 1, the tensile strength and Young's modulus of the reinforced valve leaflet 1 are significantly improved, the elongation at break of the reinforced valve leaflet 1 is reduced, and the suture force of the reinforced valve leaflet 1 is significantly increased. This indicates that the first reinforcing edge 21 made of fabric can effectively improve the mechanical properties of the material and avoid the risk of tearing at the suture portion.

In one embodiment, the preparation method for valve leaflet 1 includes the following steps:
1) obtaining a certain amount of polyurethane material (first polymer material) and heating it to 220° C. to reach a molten state;
2) cutting a PET fabric (second polymer material) into corresponding sizes through a laser cutting machine, placing the cut fabric in a mold; pouring the polyurethane material in molten state into the mold, cooling the mold slowly to room temperature to form a polymer material.
3) locating a cutting position of a cutting machine accurately, and cutting the polymer material to obtain a valve leaflet 1 as shown in FIG. 7, wherein a suture portion of the valve leaflet 1 has a first reinforcing edge 21, and a free edge 31 of the valve leaflet 1 has a second reinforcing edge 22 with a width of 0.1-5 mm, further reducing the risk of tearing at the free edge 31 of the valve leaflet 1.

As shown in FIG. 7, the width at position d is 0.1-5 mm. Generally, the stress at a center hole of the valve (i.e., position d) is relatively high. Thus, reinforcing the free edge 31, the risk of tearing of the valve leaflet 1 is reduced. Due to the small width of the reinforcement portion, it will not affect the flexibility of the valve leaflet 1.

In one embodiment, the preparation method for valve leaflet 1 includes the following steps:
1) dissolving a certain amount of polyurethane (first polymer material) in N,N-dimethylacetamide solution, heating to 80° C. and stirring for 12 hours, fully dissolving the polyurethane to obtain a polyurethane solution with a mass concentration of 12%;
2) cutting a PET fabric (second polymer material) into corresponding sizes through a laser cutting machine, placing the cut fabric in a mold and pouring the polyurethane solution into the mold; placing the mold in a blast drying oven to dry it at a temperature of 60 degrees for 12 hours;
repeating the above steps five times to obtain a polymer composite film with a thickness of 0.15 mm;
3) locating a cutting position of a cutting machine accurately to obtain locally reinforced valve leaflet 1, wherein a structure of the valve leaflet 1 is shown in FIG. 5, and the locally reinforced first reinforcing edge 21 is located at a suture portion of the valve leaflet 1;
4) placing the locally reinforced valve leaflet 1 in a plasma discharge machine for 10 minutes, using CO₂ as reaction gas, wherein a flow rate of the CO₂ is 20 cm³/min, and power of the plasma discharge machine is 180 W.

The locally reinforced valve leaflet 1 is soaked in a heparin based coating solution (or an amphiphilic coating solution, such as carboxybetaine, sulfobetaine, phosphocholine, and etc.) which contains 5 μg/ml and has a pH of 7.4. Subsequently, EDC condensing agent with a concentration of 100 mmol/L is added into the coating solution, and then the solution was placed on a shaker with a temperature of 20° C. and shaken for 2 hours. Subsequently, a fibrous membrane was removed and washed three times with PBS, thus to obtain surface modified locally reinforced valve leaflet 1.

Regarding the adhesion of the material to platelets:

Measuring the content of LDH released by platelets adsorbed on the material to detect the adhesion of the material to platelets, specific experimental process includes:
1) after reacting the sample membrane with fresh plasma rich in platelet at 37° C. for 30 minutes, cleaning the membrane carefully with PBS;
2) after natural drying in the air, adding 150 μL of LDH release reagent diluted 10 times with PBS to the well plate, shaking the 96-well plate to mix evenly, and then placing in a constant temperature incubator at 37° C. for 1 hour;
3) taking 120 μL of supernatant from each well and adding to a new 96-well plate, and then preparing a detection solution by adding three solutions (i.e., lactic acid solution, enzyme solution, and 1× INT solution) in a volume ratio of 1:1:1; adding 60 μL of detection solution to the supernatant of each well, mixing and then incubating on a horizontal shaker at room temperature in the dark for 25 minutes (incubation time is selected within the range of 20-30 minutes), and then measuring a absorbance of the solution at 490 nm using an enzyme-linked immunosorbent assay (ELISA) reader. The test results show that the surface modified polymer film has good anti-platelet adhesion performance.

The present application further provides a prosthetic valve that includes:
a stent being configured with a blood flow channel therein;
one or multiple valve leaflet 1(s) 1, the first reinforcing edge 21 being fixed to the stent, the free edge 31 of the valve leaflet 1 cooperating with an inner wall of the stent or the free edges 31 of the multiple leaflets 1 cooperating with each other to control the blood flow channel.

The stent is a tubular structure, and a tubular lumen thereof serves as the blood flow channel. Depending on the usage scenario, the stent may deform radially or not.

The prosthetic valves are not limited to aortic valves, mitral valves, tricuspid valves, pulmonary valves, and etc. The prosthetic valves with the above structural features may be used, and the valve leaflet 1s 1 for the prosthetic valves may be selected according to actual needs.

TABLE 2

| | | Test conditions | | | | | |
|---|---|---|---|---|---|---|---|
| Prosthetic valve | | | | Average | Pressure | | |
| | Size | Heart rate | Heart output | pressure | difference | EOA | Regurgitation |
| Name | (mm) | (beats/min) | (L/min) | (mmHg) | (mmHg) | (cm²) | ratio (%) |
| Control | 26 | 70 | 5 | 100 | 40 | 1.55 | 23.2 |
| Group | | | | | | | |
| Experimental | | | | | | 2.21 | 13.56 |
| Group | | | | | | | |

The specific preparation method for the experimental group includes:
1) dissolving a certain amount of polyurethane (first polymer material) in N,N-dimethylacetamide solution, heating to 80° C. and stirring for 12 hours, fully dissolving the polyurethane to prepare a polyurethane solution with a mass concentration of 12%;
2) cutting a PET fabric (second polymer material) into corresponding sizes through a laser cutting machine (as shown in FIG. 2), placing the cut fabric into a mold and pouring 0.8 mL of polyurethane solution into the mold; placing the mold in a blast drying oven to dry it at a temperature of 60 degrees for 12 hours;
repeating the above steps five times to obtain a polymer composite film with a thickness of 0.18 mm;
3) locating a cutting position of a cutting machine accurately to obtain a valve leaflet 1, wherein in the obtained locally reinforced valve leaflet 1, as shown in FIG. 5, the first reinforcing edge 21 is located at a suture portion of the valve leaflet 1, and the free edge 31 of the valve leaflet 1 has no reinforcing fabric;
4) suturing the valve leaflet 1 onto the stent to obtain a 29 mm self-expanding polymer valve.

The difference between the control group and the experimental group is that all portions of the valve leaflet 1 are reinforced with fabric.

According to Table 2, the effective orifice area of the prosthetic valve prepared with locally reinforced valve leaflet 1 is increased, and the regurgitation ratio of the prosthetic valve is reduced, which indicates that the locally reinforced valve leaflet 1 is softer and more flexible. At the same time, the free edge 31 of the valve leaflet 1 has a high degree of fit and less regurgitation. For fatigue resistance test: the prosthetic valve prepared using locally reinforced valve leaflet 1 has a fatigue resistance of 400 million times, while the unreinforced polymer valve has a fatigue resistance of 20 million times and tears occur at the suture site. Note: the first polymer material selected for the above two valves is the same.

The various technical features of the above embodiments may be combined in any way. In order to make the description concise, not all possible combinations of the various technical features in the above embodiments have been described. However, as long as there is no contradiction in the combination of these technical features, they should be considered within the scope of this specification.

The above-described embodiments merely express several implementation manners of the present application, and the description thereof is relatively specific and detailed, but it should not be construed as limiting the scope of the patent application. It should be pointed out that, for ordinary technicians in this field, modifications and improvements can be made without departing from the concept of the present application, which all fall within the scope of protection of the present application.

What is claimed is:

1. A valve leaflet for prosthetic valve, comprising:
a main body, an edge portion of the main body comprising a free edge and a fixing edge that are opposite to each other, the main body being made of a first polymer material;
a first reinforcing edge distributed along the fixing edge, the first reinforcing edge being made of a second polymer material which is configured with a porous structure, the first polymer material entering the porous structure of the first reinforcing edge and being fixed to the first reinforcing edge.

2. The valve leaflet for prosthetic valve according to claim 1, further comprising:
a second reinforcing edge distributed along the free edge, the second reinforcing edge being made of a third polymer material which is configured with a porous structure, the first polymer material entering the porous structure of the second reinforcing edge and being fixed to the second reinforcing edge.

3. The valve leaflet for prosthetic valve according to claim 2, wherein
the first reinforcing edge and the second reinforcing edge are made of the same material; or the first reinforcing edge and the second reinforcing edge are integrally formed as one piece.

4. The valve leaflet for prosthetic valve according to claim 2, wherein
the second reinforcing edge in whole is substantially strip-shaped and extends along the free edge with a constant width.

5. The valve leaflet for prosthetic valve according to claim 2, wherein
the first reinforcing edge is configured as a sheet, with a uniform thickness of 0.01-0.3 mm;
and, the second reinforcing edge is configured as a sheet, with a uniform thickness of 0.01-0.3 mm.

6. The valve leaflet for prosthetic valve according to claim 5, wherein
the first reinforcing edge is configured as a sheet, with a uniform thickness of 0.05˜0.2 mm; and, the second reinforcing edge is configured as a sheet, with a uniform thickness of 0.05˜0.2 mm.

7. The valve leaflet for prosthetic valve according to claim 2, wherein
the valve leaflet comprises a first region avoiding the first reinforcing edge and/or second reinforcing edge, and a second region overlapping the first reinforcing edge and/or second reinforcing edge, and the first and second regions are substantially the same in thickness.

8. The valve leaflet for prosthetic valve according to claim 2, wherein
the first polymer material is at least one of polyurethane, polyolefin, and polysiloxane;
the second polymer material is configured in form of a fabric, and a raw material of the second polymer material is one of polyester, aramid, nylon, silk, and polyurethane; and
the third polymer material is configured in form of a fabric, and a raw material of the third polymer material is one of polyester, aramid, nylon, silk, and polyurethane; and
wherein the fabric is non-woven fabric or woven fabric.

9. The valve leaflet for prosthetic valve according to claim 8, wherein
the fabric is a woven fabric with a weft knitted structure and a porosity of 30-80%.

10. The valve leaflet for prosthetic valve according to claim 8, wherein
the first polymer material, the second polymer material, and the third polymer material are prepared using the same type of monomer.

11. The valve leaflet for prosthetic valve according to claim 2, wherein
the first polymer material is polyurethane, with a number average molecular weight of 30000-200000 and a hard segment content of 35-50%; and
the second polymer material and the third polymer material both are polyurethane, with a number average molecular weight of 80000-250000 and a hard segment content of 45-60%, and the second polymer material and the third polymer material both are configured in form of non-woven fabric and prepared by electrostatic spinning.

12. The valve leaflet for prosthetic valve according to claim 2, wherein
the first reinforcing edge extends outward from a position thereof adjacent to the free edge to form a hanging ear that is sutured with a stent.

13. The valve leaflet for prosthetic valve according to claim 2, wherein
a thickness of the valve leaflet is 0.07-0.5 mm.

14. A preparation method for valve leaflet, comprising:
providing a reinforcing material configured in sheet, the reinforcing material having a porous structure and a reserved region;
compositing a first polymer material in flowing state with the reinforcing material; and
solidifying the first polymer material, and cutting the reinforcing material and/or solidified first polymer material according to a predetermined shape to obtain a valve leaflet, wherein a portion of the valve leaflet with the reinforcing material is a reinforcing edge;
wherein, the reserved region is a hollow portion of the reinforcing material, and an edge of the hollow portion is partially open or a closed curve.

15. The preparation method for valve leaflet according to claim 14, wherein the edge of the hollow portion is closed curve, and the reinforcing material comprises a first reinforcing region and a second reinforcing region enclosing the reserved region,
the first reinforcing region being configured with one side thereof facing the reserved region and corresponding to the fixing edge of the valve leaflet and another side thereof extending away from the reserved region; and
the second reinforcing region being configured with one side thereof facing the reserved region and corresponding to the free edge of the valve leaflet and another side thereof extending away from the reserved region.

16. The preparation method for valve leaflet according to claim 15, wherein
the first reinforcing region and the second reinforcing region are woven together using a second polymer material and then cut out to form the reserved region; or,
the first reinforcing region and the second reinforcing region are woven together and avoided the reserved region during the weaving process.

17. The preparation method for valve leaflet according to claim 14, wherein the first polymer material in flowing state comprises a solution state or a molten state, and has a viscosity of 0.4-2.0 Pa·s; and wherein
the first polymer material in molten state has a melting temperature of 150-300° C.

18. A valve leaflet for prosthetic valve, wherein
the valve leaflet is formed by the preparation method of claim 14.

19. A prosthetic valve, comprising:
a stent being configured with a blood flow channel therein;
at least one valve leaflet of claim 18, wherein the first reinforcing edge of the valve leaflet is fixed to the stent, and the free edge of the valve leaflet cooperates with an inner wall of the stent or the free edges of multiple leaflets cooperate with each other to control the blood flow channel.

20. A prosthetic valve, comprising:
a stent being configured with a blood flow channel therein;
at least one valve leaflet of claim 1, wherein the first reinforcing edge of the valve leaflet is fixed to the stent, and the free edge of the valve leaflet cooperates with an inner wall of the stent or the free edges of multiple leaflets cooperate with each other to control the blood flow channel.
